(19) United States
(12) Patent Application Publication
(10) Pub. No.: US 2020/0268779 A1
(43) Pub. Date: Aug. 27, 2020

(54) USE OF SECOISOLARICIRESINOL DIGLUCOSIDES (SDGs) AND RELATED COMPOUNDS FOR PROTECTION AGAINST RADIATION-INDUCED CARDIOVASCULAR DYSFUNCTION

(71) Applicant: THE TRUSTEES OF THE UNIVERSITY OF PENNSYLVANIA, Philadelphia, PA (US)

(72) Inventor: Melpo CHRISTOFIDOU-SOLOMIDOU, Eagleville, PA (US)

(73) Assignee: THE TRUSTEES OF THE UNIVERSITY OF PENNSYLVANIA, Philadelphia, PA (US)

(21) Appl. No.: 16/651,145

(22) PCT Filed: Sep. 27, 2018
(86) PCT No.: PCT/US18/53139

§ 371 Date: Mar. 26, 2020

Related U.S. Application Data
(60) Provisional application No. 62/564,153, filed on Sep. 27, 2017.

(51) Int. Cl. A61K 31/7032

(52) U.S. Cl. CPC A61K 31/7032; A61K 9/0053

(57) ABSTRACT

The invention relates to the use of secoisolariciresinol diglucoside (SDG), obtained from natural sources, such as flaxseed, or generated synthetically (synthetic SDG is also referred to herein as LGM2605), other active components in flaxseed, secoisolariciresinol (SECO), enterodiol (ED), and enterolactone (EL), as well as stereoisomers of the foregoing, metabolites of the foregoing, degradants of the foregoing, and analogs of the foregoing, to treat and protect cardiac and vascular tissues, for example, treating ionizing radiation-associated vascular injury and vasculopathy and protecting vascular tissue against ionizing radiation exposure.

### GOVERNMENT INTEREST STATEMENT

This invention was made with U.S. government support under grant numbers HHSN272201500005C and BAA-NIAID-DAIT-NIHAI2014001 from the National Institutes of Health. The government has certain rights in the invention.

### FIELD OF THE INVENTION

The invention relates to the use of secoisolariciresinol diglucoside (SDG), obtained from natural sources, such as flaxseed, or generated synthetically (synthetic SDG is also referred to herein as LGM2605), other active components in flaxseed, secoisolariciresinol (SECO), enterodiol (ED), and enterolactone (EL), as well as stereoisomers of the foregoing, metabolites of the foregoing, degradants of the foregoing, and analogs of the foregoing, to treat and protect cardiac and vascular tissues, for example, treating ionizing radiation-associated vascular injury and vasculopathy and protecting vascular tissue against ionizing radiation exposure.

### BACKGROUND OF THE INVENTION

Ionizing radiation produces a wide range of deleterious effects in living organisms. Humans are exposed to radiation as an occupational hazard, during diagnostic and therapeutic radiographic procedures, when using electronic devices, from background radiation of nuclear accidents, during air and space travel, as well as from prolonged exposure to the sun (e.g., sun bathers or outdoor workers). Exposure to natural radiation can occur in many forms: natural resources such as air, water, and soil may become contaminated when it comes in contact with naturally-occurring, radiation-emitting substances (radionuclides); radon is one such common source of natural radiation. Current global developments have additionally established terrorism as a dangerous means by which potentially large numbers of people can be exposed to lethal amounts of radiation. It is, therefore, of high importance to identify agents that can be administered before and during radiation exposure (i.e., radioprotective agents), and as treatment after radiation exposure (i.e., radiation mitigators).

Radiation therapy involves the use of high-energy waves or streams, commonly referred to as radiation, that induce ionization in tissue to treat disease. Radiation therapy may be referred to by such terms a radiotherapy, x-ray therapy, gamma-ray therapy, beta-ray therapy, charged-particle therapy, or irradiation.

In a number of cases, normal cells damaged during radiation exposure will recover, but in some cases, they will not. One complication of radiation exposure (e.g., during treatment) is radiation-induced vasculopathy. The vasculature, in particular vascular endothelial cells, is very sensitive to radiation (Mouthon et al., Radiat Res (2003) 160:593; Milliat et al., Am J. Pathol (2006) 169:1484; Garcia et al., Science (2003) 300:1155). Following injury, vascular networks undergo a progressive fibrous obliteration, resulting in a loss of perfusion, ischemia, and tissue necrosis. Some radiation therapies that may cause radiation-induced vasculopathy include plaque brachytherapy, external beam irradiation, or proton beam.

Radiation exposure can also affect the heart proper. It has been shown that exposure of the heart to ionizing radiation, severely increases the risk of heart failure and that there are cardiac pathologies that are linked to radiation exposure (Yusuf et al., Radiation-Induced Heart Disease: A Clinical Update, Cardiology Research and Practice, 2011; see also Donnellan et al., Radiation-induced heart disease: A Practical Guide to Diagnosis and Management, Cleveland Clinic Journal of Medicine. 2016; Vol. 83(12) pp.914-922). Thus, radiation therapy can cause injury to all the components of the heart, including damage to the small vessels that supply blood to the heart. It may also cause scarring in the heart muscle, and coronary arteries are more prone to clotting after being treated with radiation. The radiation may damage the endothelial lining of the vessels making them form clots more readily. This damage renders the heart unable to efficiently pump blood throughout the body. Symptoms of this effect include shortness of breath, fatigue, and anemia.

Lung cancer remains the leading cause of cancer deaths in the US and worldwide. Introduction of proton radiation therapy, led to reducing the dose received by normal tissue surrounding the tumor, while allowing more focused doses to the tumor target. Nevertheless, a substantial risk of late side effects in long term survivors, such as significant normal tissue damage, still remains.

### SUMMARY OF THE INVENTION

In one aspect, provided herein are methods for treating or preventing vascular injury and/or for protecting vascular tissue in a subject in need thereof, comprising: administering to the subject an effective amount of secoisolaricirecinol diglucoside (SDG), an analog thereof, a stereoisomer thereof, or a combination thereof. In another aspect, provided herein are methods for treating or preventing injury to cardiac tissue and/or for protecting cardiac tissue in a subject in need thereof, comprising: administering to the subject an effective amount of secoisolaricirecinol diglucoside (SDG), an analog thereof, a stereoisomer thereof, or a combination thereof.

In another aspect, provided herein are methods for treating or preventing radiation-associated vascular injury in a subject in need thereof, comprising: administering to the subject an effective amount of secoisolaricirecinol diglucoside (SDG), an analog thereof, a stereoisomer thereof, or a combination thereof, thereby treating said radiation-induced vascular damage in the subject.

In another aspect, provided herein are methods for improving arterial blood oxygenation in a subject who has been or will be exposed to ionizing radiation, comprising: administering to the subject an effective amount of SDG, an analog thereof, a stereoisomer thereof, or a combination thereof, thereby improving arterial blood oxygenation in the subject.

In a further aspect, provided herein are methods for protecting vascular tissue against ionizing radiation exposure in a subject in need thereof, comprising: administering to the subject an effective amount of SDG, an analog thereof, a stereoisomer thereof, or a combination thereof, thereby protecting normal vascular tissue against ionizing radiation exposure in the subject.

In an additional aspect, provided herein are methods for reducing or preventing ionizing radiation-associated blood vessel wall scarring in a subject in need thereof, comprising: administering to the subject an effective amount of SDG, an analog thereof, a stereoisomer thereof, or a combination thereof, thereby reducing or preventing ionizing radiation-associated blood vessel wall scarring in the subject.

### BRIEF DESCRIPTION OF THE DRAWINGS

FIG. 1. Arterial Oxygen saturation (A) and Pulse Distention (B) were measured in living mice (n=5), between 130 and 140 days post single fraction thoracic radiation exposure.

### DETAILED DESCRIPTION OF THE INVENTION

The invention relates to the use of secoisolariciresinol diglucoside (SDG), obtained from natural sources, such as flaxseed, or generated synthetically (synthetic SDG is also referred to herein as LGM2605), other active components in flaxseed, and related compounds, to treat and protect cardiac and vascular tissues, for example, treating ionizing radiation-associated cardiovascular injury and protecting normal vascular tissue against ionizing radiation exposure.

For example, in some embodiments, secoisolariciresinol diglucoside (SDG), obtained from natural sources, such as flaxseed, or generated synthetically, other active components in flaxseed, and related compounds are used to treat ionizing radiation-associated vascular injury and to protect normal vascular tissue against ionizing radiation exposure. Surprisingly and unexpectedly, the inventors have found that SDG can treat proton radiation-associated vascular injury and protect vascular tissue against proton radiation exposure.

Accordingly, in one aspect, provided herein are methods for treating or preventing vascular injury and/or for protecting vascular tissue in a subject in need thereof, comprising: administering to the subject an effective amount of secoisolaricirecinol diglucoside (SDG), an analog thereof, a stereoisomer thereof, or a combination thereof. In another aspect, provided herein are methods for treating or preventing injury to cardiac tissue and/or for protecting cardiac tissue in a subject in need thereof, comprising: administering to the subject an effective amount of secoisolaricirecinol diglucoside (SDG), an analog thereof, a stereoisomer thereof, or a combination thereof.

More specifically, provided herein are methods for treating or preventing radiation-associated cardiovascular injury in a subject in need thereof, comprising: administering to the subject an effective amount of SDG, an analog thereof, a stereoisomer thereof, or a combination thereof.

In another embodiment, provided herein are methods for treating or preventing radiation-associated cardiac injury in a subject in need thereof, comprising: administering to the subject an effective amount of SDG, an analog thereof, a stereoisomer thereof, or a combination thereof. Also provided herein are methods for treating or preventing radiation-associated vascular injury in a subject in need thereof, comprising: administering to the subject an effective amount of SDG, an analog thereof, a stereoisomer thereof, or a combination thereof.

Histopathology of radiation-induced vasculopathy reveals both the destruction of vascular endothelial cells and pericytes. Radiation vasculopathy includes such damage as obliterative endarteritis (endothelial cell loss and thickened vessel walls), and pulmonary microangiopathy. This damage presents as tissue ischemia caused by undesirable conditions such as vascular occlusions, capillary dropout and leakage (hemorrhages, exudation and edema) and microaneurysms. If these undesirable conditions are left untreated, they may worsen and/or cause other undesirable conditions that ultimately lead to a loss of organ function (e.g., where the organ is a lung, loss of pulmonary vasculature function presents as the symptoms of decline of arterial blood oxygenation, increase of pulmonary blood vessel wall thickness, collagen scarring of pulmonary vasculature, and loss of pulmonary blood vessel wall flexibility and distensibility).

Without wishing to be bound by theory, on a molecular level certain cytokines and growth factors, such as TGF-β1 and IL-1β, may stimulate radiation-induced endothelial proliferation, fibroblast proliferation, collagen deposition, and fibrosis leading to advanced lesions of atherosclerosis.

Thus, in one embodiment, the radiation-associated vascular injury treated by compositions described herein is obliterative endarteritis. In another embodiment, the radiation-associated vascular injury treated by compositions described herein is pulmonary microangiopathy. In a further embodiment, the radiation-associated vascular injury treated by compositions described herein is a vascular occlusion. In another embodiment, the radiation-associated vascular injury treated by compositions described herein is capillary dropout and leakage. In another embodiment, the radiation-associated vascular injury treated by compositions described herein is a microaneurysm.

In another embodiment, the radiation-associated vascular injury treated by compositions described herein is cardiac ischemia. In another embodiment, the radiation-associated vascular injury treated by compositions described herein is coronary artery disease. In another embodiment, the radiation-associated vascular injury treated by compositions described herein is valvular disease.

In one embodiment, provided herein are methods for preventing radiation-associated cardiovascular injury in a subject in need thereof, comprising: administering to the subject an effective amount of secoisolaricirecinol diglucoside (SDG), an analog thereof, a stereoisomer thereof, or a combination thereof. In another embodiment, provided herein are methods for preventing radiation-associated cardiac injury in a subject in need thereof, comprising: administering to the subject an effective amount of SDG, an analog thereof, a stereoisomer thereof, or a combination thereof. In another embodiment, provided herein are methods for preventing radiation-associated vascular injury in a subject in need thereof, comprising: administering to the subject an effective amount of SDG, an analog thereof, a stereoisomer thereof, or a combination thereof.

In an additional embodiment, provided herein are methods for protecting vascular tissue against ionizing radiation exposure in a subject in need thereof, comprising: administering to the subject an effective amount of SDG, an analog thereof, a stereoisomer thereof, or a combination thereof.

In a further embodiment, provided herein are methods for reducing, suppressing, or reversing symptoms of radiation-associated vascular injury in a subject in need thereof, comprising: administering to the subject an effective amount of SDG, an analog thereof, a stereoisomer thereof, or a combination thereof.

The symptoms of radiation-associated vascular injury include, without limitation, decline of arterial blood oxygenation, increase of blood vessel wall thickness, perivascular fibrosis, collagen scarring of pulmonary vasculature, and loss of blood vessel wall flexibility and distensibility.

Thus, in another embodiment, provided herein are methods for improving arterial blood oxygenation in a subject who has been or will be exposed to ionizing radiation, comprising: administering to the subject an effective amount of secoisolaricirecinol diglucoside (SDG), an analog thereof, a stereoisomer thereof, or a combination thereof, thereby improving arterial blood oxygenation in the subject.

In a further embodiment, provided herein are methods for reducing or preventing ionizing radiation-associated blood vessel wall scarring in a subject in need thereof, comprising: administering to the subject an effective amount of SDG, an analog thereof, a stereoisomer thereof, or a combination thereof, thereby reducing or preventing ionizing radiation-associated blood vessel wall scarring in the subject.

In a yet further embodiment, provided herein are methods for reducing or preventing ionizing radiation-associated perivascular fibrosis in a subject in need thereof, comprising: administering to the subject an effective amount of SDG, an analog thereof, a stereoisomer thereof, or a combination thereof, thereby reducing or preventing ionizing radiation-associated perivascular fibrosis in the subject.

In an additional embodiment, provided herein are methods for maintaining blood vessel distensibility following ionizing radiation exposure in a subject in need thereof, comprising: administering to the subject an effective amount of SDG, an analog thereof, a stereoisomer thereof, or a combination thereof, thereby maintaining blood vessel distensibility following ionizing radiation exposure in the subject.

2,3-bis (3-methoxy-4-hydroxybenzyl) butane-1,4-diol (secoisolariciresinol or SECO) is the primary lignan found in flaxseed. In its native state, it is stored in the plant as the conjugate SDG. Flaxseed, its bioactive ingredients, and its metabolites are known in the art and described in U.S. Publ. Nos: 2010/0239696; 2011/0300247; and 2014/0308379; and in International Publ. No. WO2014/200964, each of which is incorporated by reference herein in its entirety.

SDG can be isolated from natural sources or chemically synthesized. Due to complex extraction, purification and enrichment methods to isolate secoisolariciresinol diglucoside (SDG) from natural resources, in a preferred embodiment, SDG is chemically synthesized.

Techniques for synthesizing SDG, its stereoisomers and analogs are described in Mishra et al., Bioorganic & Medicinal Chemistry Letters (2013) 19:5325 and in International Patent Publ. No. WO2014/200964, each of which is hereby incorporated by reference in its entirety. For example, using the natural compounds vanillin and glucose, two enantiomers (their structures are depicted below) of SDG: SDG (S,S) and SDG (R,R), were successfully synthesized (Mishra et al., Bioorganic & Medicinal Chemistry Letters 2013, (19):5325).

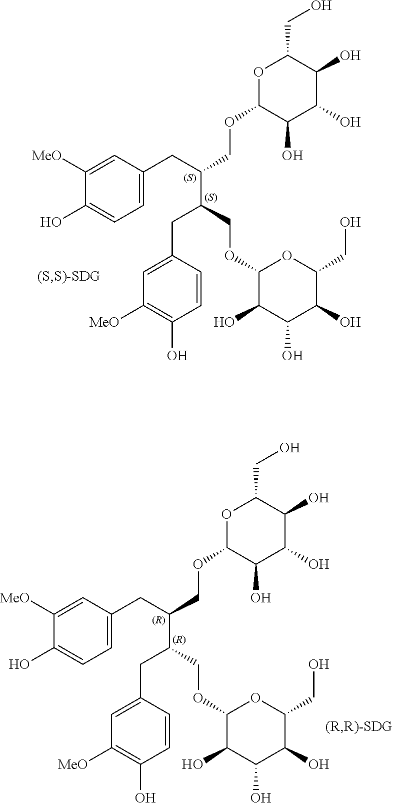

In one embodiment, the SDG administered in the methods described herein is SDG (S,S). In another embodiment, the SDG administered in the methods described herein is SDG (R,R).

SDG is metabolized in the human intestine to enterodiol (ED), and enterolactone (EL). Synthetic analogs of enterodiol and enterolactone are known (see, e.g., Eklund et al., Org. Lett., 2003, 5:491). Thus, in another aspect, other bioactive ingredients of flaxseed, their metabolites, their degradants or stereoisomers can also be used. Examples of the other bioactive ingredients of flaxseed include, but not limited to, secoisolariciresinol (SECO), enterodiol (ED), enterolactone (EL), analogs thereof, isomers (including stereoisomers) thereof, or a combination thereof.

Bioactive components for use in the methods provided herein may also be chemically synthesized directly into the mammalian, readily metabolizable forms, Enterodiol (ED) or Enterolactone (EL), as is known in the art.

In another aspect, provided herein are methods for treating or preventing vascular injury and/or for protecting vascular tissue in a subject in need thereof, comprising: administering to the subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof. In another aspect, provided herein are methods for treating or preventing injury to cardiac tissue and/or for protecting cardiac tissue in a subject in need thereof, comprising: administering to the subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

Thus, in one embodiment, provided herein are methods for treating or preventing radiation-associated cardiovascular damage in a subject who has been or will be exposed to radiation, comprising: administering to the subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof. In another embodiment, provided herein are methods for treating or preventing radiation-associated vascular damage in a subject who has been or will be exposed to radiation, comprising: administering to the subject an effective amount of at least one bioactive ingredient, wherein said bioactive ingredient comprises SDG, SECO, ED, EL, metabolites thereof, degradants thereof, analogs thereof, stereoisomers thereof, or a combination thereof.

In another embodiment, provided herein are methods for treating or preventing ionizing radiation-associated vascular injury in a subject in need thereof, comprising: administering to the subject an effective amount of at least one bioactive ingredient, wherein said bioactive ingredient comprises SDG, SECO, ED, EL, metabolites thereof, degradants thereof, analogs thereof, stereoisomers thereof, or a combination thereof. In a further embodiment, provided herein are methods for protecting vascular tissue against ionizing radiation exposure in a subject in need thereof, comprising: administering to the subject an effective amount of at least one bioactive ingredient, wherein said bioactive ingredient comprises SDG, SECO, ED, EL, metabolites thereof, degradants thereof, analogs thereof, stereoisomers thereof, or a combination thereof.

In a yet further embodiment, provided herein are methods for reducing, suppressing, or reversing symptoms of radiation-associated vascular injury in a subject in need thereof, comprising: administering to the subject an effective amount of at least one bioactive ingredient, wherein said bioactive ingredient comprises SDG, SECO, ED, EL, metabolites thereof, degradants thereof, analogs thereof, stereoisomers thereof, or a combination thereof.

In an additional embodiment, provided herein are methods for improving arterial blood oxygenation in a subject who has been or will be exposed to ionizing radiation, comprising: administering to the subject an effective amount of at least one bioactive ingredient, wherein said bioactive ingredient comprises secoisolaricirecinol digluco side (SDG), secoisolaricirecinol (SECO), enterodiol (ED), enterolactone (EL), metabolites thereof, degradants thereof, analogs thereof, stereoisomers thereof, or a combination thereof. In another embodiment, provided herein are methods for reducing or preventing ionizing radiation-associated blood vessel wall scarring in a subject in need thereof, comprising: administering to the subject an effective amount of at least one bioactive ingredient, wherein said bioactive ingredient comprises SDG, SECO, ED, EL, metabolites thereof, degradants thereof, analogs thereof, stereoisomers thereof, or a combination thereof. In another embodiment, provided herein are methods for reducing or preventing ionizing radiation-associated perivascular fibrosis in a subject in need thereof, comprising: administering to the subject an effective amount of at least one bioactive ingredient, wherein said bioactive ingredient comprises SDG, SECO, ED, EL, metabolites thereof, degradants thereof, analogs thereof, stereoisomers thereof, or a combination thereof.

In another embodiment, provided herein are methods for maintaining blood vessel distensibility following ionizing radiation exposure in a subject in need thereof, comprising: administering to the subject an effective amount of at least one bioactive ingredient, wherein said bioactive ingredient comprises SDG, SECO, ED, EL, metabolites thereof, degradants thereof, analogs thereof, stereoisomers thereof, or a combination thereof. In another embodiment, provided herein are methods for treating or preventing radiation-associated cardiac damage in a subject who has been or will be exposed to radiation, comprising: administering to the subject an effective amount of at least one bioactive ingredient, wherein said bioactive ingredient comprises SDG, SECO, ED, EL, metabolites thereof, degradants thereof, analogs thereof, stereoisomers thereof, or a combination thereof.

In another aspect, flaxseed extract can be used. Techniques for extracting and purifying SDG are known in the art and described in U.S. Pat. No. 5,705,618, which is incorporated herein by reference in its entirety.

Thus, in one embodiment, provided herein are methods for treating or preventing vascular injury and/or for protecting vascular tissue in a subject in need thereof, comprising: administering to the subject an effective amount of a flax seed extract. In another embodiment, provided herein are methods for treating or preventing injury to cardiac tissue and/or for protecting cardiac tissue in a subject in need thereof, comprising: administering to the subject an effective amount of a flax seed extract.

In one embodiment, provided herein are methods for treating or preventing radiation-associated cardiovascular damage in a subject who has been or will be exposed to radiation, comprising: administering to the subject an effective amount of a flax seed extract. In another embodiment, provided herein are methods for treating or preventing ionizing radiation-associated vascular injury in a subject in need thereof, comprising: administering to the subject an effective amount of a flax seed extract. In another embodiment, provided herein are methods for protecting vascular tissue against ionizing radiation exposure in a subject in need thereof, comprising: administering to the subject an effective amount of a flax seed extract. In a further embodiment, provided herein are methods for reducing, suppressing, or reversing symptoms of radiation-associated vascular injury in a subject in need thereof, comprising: administering to the subject an effective amount of a flax seed extract. In a further embodiment, provided herein are methods for improving arterial blood oxygenation in a subject who has been or will be exposed to ionizing radiation, comprising: administering to the subject an effective amount of a flax seed extract.

In a further embodiment, provided herein are methods for reducing or preventing ionizing radiation-associated blood vessel wall scarring in a subject in need thereof, comprising: administering to the subject an effective amount of a flax seed extract. In another embodiment, provided herein are methods for reducing or preventing ionizing radiation-associated perivascular fibrosis in a subject in need thereof, comprising: administering to the subject an effective amount of a flax seed extract. In a yet further embodiment, provided herein are methods for maintaining blood vessel distensibility following ionizing radiation exposure in a subject in need thereof, comprising: administering to the subject an effective amount of a flax seed extract. Thus, in one embodiment, provided herein are methods for treating or preventing radiation-associated cardiac damage in a subject who has been or will be exposed to radiation, comprising: administering to the subject an effective amount of a flax seed extract.

In one embodiment, the blood vessel is an artery. In another embodiment, the blood vessel is a vein. In another embodiment, the blood vessel is a capillary.

In one embodiment, the blood vessel is a part of coronary vasculature. In another embodiment, the blood vessel is a part of brain vasculature. In another embodiment, the blood vessel is a part of limb vasculature. In another embodiment, the blood vessel is a part of hepatic vasculature. In another embodiment, the blood vessel is any blood vessel known in the art. In a preferred embodiment, the blood vessel is a part of pulmonary vasculature.

A “metabolite” is a substance produced by metabolism or by a metabolic process. For example, a metabolite of SDG is EL or ED. A “degradant” is a product of the breakdown of a molecule, such as SDG, into smaller molecules. It will be appreciated by one skilled in the art that a metabolite or a degradant may be a chemically synthesized equivalent of a natural metabolite or degradant.

An “analog” is a compound whose structure is related to that of another compound. The analog may be a synthetic analog.

An “ingredient” or “component” is an element or a constituent in a mixture or compound.

In another aspect, the invention relates to a pharmaceutical composition. “Pharmaceutical composition” refers to an effective amount of an active ingredient, e.g., (S,S)-SDG (R,R)-SDG, meso-SDG, SDG, SECO, EL, ED and analogs thereof, together with a pharmaceutically acceptable carrier or diluent.

The compositions described herein may include a “therapeutically effective amount.” A “therapeutically effective amount” refers to an amount effective, at dosages and for periods of time necessary, to achieve the desired therapeutic result. A therapeutically effective amount may vary according to factors such as the disease state, age, sex, and weight of the individual, and the ability of the composition to elicit a desired response in the individual. A therapeutically effective amount is also one in which toxic or detrimental effects of the molecule are outweighed by the therapeutically beneficial effects.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used herein includes both one and more than one such excipient.

The pharmaceutical compositions can be administered to a subject by any suitable method known to a person skilled in the art, such as orally, parenterally, transmuco sally, transdermally, intramuscularly, intravenously, intra-dermally, subcutaneously, intra-peritonealy, intra-ventricularly, intra-cranially, intra-vaginally, intra-tumorally, or bucally. Controlled release may also be used by embedding the active ingredient in an appropriate polymer which may then be inserted subcutaneously, intratumorally, bucally, as a patch on the skin, or vaginally. Coating a medical device with the active ingredient is also covered.

In some embodiments, the pharmaceutical compositions are administered orally, and are thus formulated in a form suitable for oral administration, i.e., as a solid or a liquid preparation. Suitable solid oral formulations include tablets, capsules, pills, granules, pellets and the like. Suitable liquid oral formulations include solutions, suspensions, dispersions, emulsions, oils and the like. In some embodiments, the active ingredient is formulated in a capsule. In accordance with this embodiment, the compositions described herein comprise, in addition to the active compound and the inert carrier or diluent, drying agent, in addition to other excipients as well as a gelatin capsule. In one embodiment, the compositions described herein are administered in a dietary composition.

In some embodiments, the pharmaceutical compositions are administered by intravenous, intra-arterial, or intra-muscular injection of a liquid preparation. In some embodiments, the pharmaceutical composition is a liquid preparation formulated for oral administration. In some embodiments, the pharmaceutical composition is a liquid preparation formulated for intravaginal administration. Suitable liquid formulations include solutions, suspensions, dispersions, emulsions, oils and the like. In some embodiments, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In some embodiments, the pharmaceutical compositions are administered intra-muscularly and are thus formulated in a form suitable for intra-muscular administration. In some embodiments, the pharmaceutical compositions are administered intra-bucally and are thus formulated in a form suitable for buccal administration.

In some embodiments, the pharmaceutical compositions are administered topically to body surfaces and are thus formulated in a form suitable for topical administration. Suitable topical formulations include gels, ointments, creams, lotions, drops, controlled release polymers and the like. For topical administration, the flaxseed, its bioactive ingredient, or a metabolite thereof is prepared and applied as a solution, suspension, or emulsion in a physiologically acceptable diluent with or without a pharmaceutical carrier.

In some embodiments, the pharmaceutical compositions provided herein are controlled-release compositions, i.e. compositions in which the flaxseed, its bioactive ingredient, or a metabolite thereof is released over a period of time after administration. Controlled- or sustained-release compositions include formulation in lipophilic depots (e.g. fatty acids, waxes, oils). In other embodiments, the composition is an immediate-release composition, i.e. a composition in which all the flaxseed, its bioactive ingredient, or a metabolite thereof is released immediately after administration.

In some embodiments, compositions for use in the methods provided herein are administered at a therapeutic dose once per day. In some embodiments, the compositions are administered once every two days, twice a week, once a week, or once every two weeks.

In one embodiment, (S,S)-SDG (R,R)-SDG, (S,R)-SDG (R,S)-SDG, meso-SDG, SECO, EL, ED or an analog thereof may be administered at a dose of 0.1 ng/kg to 500 mg/kg. In another embodiment, (S,S)-SDG (R,R)-SDG, (S,R)-SDG (R,S)-SDG, meso-SDG, SECO, EL, ED or an analog thereof may be administered at a concentration of about 1 nanomolar (nM) to about 1 molar (M). In another embodiment, (S,S)-SDG (R,R)-SDG, (S,R)-SDG (R,S)-SDG, meso-SDG, SECO, EL, ED or an analog thereof may be administered at a concentration from about 25 μM to about 250 μM.

The treatment regimen with (S,S)-SDG (R,R)-SDG, (S,R)-SDG (R,S)-SDG, meso-SDG, SDG, SECO, EL, ED or an analog thereof may range from a single administration to several days, months, years, or indefinitely. In one embodiment, treatment regimen with (S,S)-SDG (R,R)-SDG, (S,R)-SDG (R,S)-SDG, meso-SDG, SDG, SECO, EL, ED or an analog thereof comprises daily administration over one week. In another embodiment, treatment regimen with (S,S)-SDG (R,R)-SDG, (S,R)-SDG (R,S)-SDG, meso-SDG, SDG, SECO, EL, ED or an analog thereof comprises daily administration over two weeks. In another embodiment, treatment regimen with (S,S)-SDG (R,R)-SDG, (S,R)-SDG (R,S)-SDG, meso-SDG, SDG, SECO, EL, ED or an analog thereof comprises daily administration over three weeks. In another embodiment, treatment regimen with (S,S)-SDG (R,R)-SDG, (S,R)-SDG (R,S)-SDG, meso-SDG, SDG, SECO, EL, ED or an analog thereof comprises daily administration over one month. In another embodiment, treatment regimen with (S,S)-SDG (R,R)-SDG, (S,R)-SDG (R,S)-SDG, meso-SDG, SDG, SECO, EL, ED or an analog thereof comprises daily administration over two months. In another embodiment, treatment regimen with (S,S)-SDG (R,R)-SDG, (S,R)-SDG (R,S)-SDG, meso-SDG, SDG, SECO, EL, ED or an analog thereof comprises daily administration over three months. In another embodiment, treatment regimen with (S,S)-SDG (R,R)-SDG, (S,R)-SDG (R,S)-SDG, meso-SDG, SDG, SECO, EL, ED or an analog thereof comprises daily administration over six months.

As used herein, the term “vascular” is used broadly to refer to the circulatory system of an organism. As such, the term “vascular” refers to arteries and veins, as well as specialized organs that are closely associated with the circulatory system, such as the heart. The term “cardiovascular” refers to that portion of the vascular system that is closely associated with the heart.

As used herein, “treating” may refer to either therapeutic treatment or prophylactic or preventative measures, where the object is to prevent or lessen the targeted pathologic condition or disorder as described herein, or both. Therefore, compositions for use in the methods provided herein may be administered to a subject before exposure, e.g., to radiation, a carcinogen, a toxicant, or hypochlorite ions. In some cases, compositions for use in the methods provided herein may be administered to a subject after exposure. Thus, treating a condition as described herein may refer to preventing, inhibiting, or suppressing the condition in a subject.

Furthermore, as used herein, the terms “treat” and “treatment” refer to therapeutic treatment, as well prophylactic or preventative measures, where the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already having been exposed, e.g., to radiation, a carcinogen, a toxicant, or hypochlorite ions, as well as those prone to being exposed or those expecting to be exposed.

As used herein, the term “preventing” may refer to stopping, hindering, or suppressing a disease, disorder, or a symptom of a disease or disorder, through some action before the symptoms or consequences of the disease or disorder manifest themselves, or before a patient is exposed to conditions which may trigger the disease or disorder.

The diseases or conditions that can be treated or prevented by the compositions of the invention include, but are not limited to, vascular ionizing radiation damage (e.g., proton radiation damage in pulmonary vasculature) and cardiac ionizing radiation damage.

In one example, subjects in need of radioprotection or radiation mitigation according to methods provided herein are subjects who will, are, or have been exposed to potentially deleterious amounts of radiation. It will be understood that such exposure may be a single exposure, periodic exposure, sporadic exposure or ongoing exposure to the radiation. It is also understood that such radiation exposure includes accidental exposure, incidental or intentional exposure.

Thus, in one embodiment, the compositions described herein are administered prior to exposure to ionizing radiation. In another embodiment, the compositions described herein are administered concurrently with exposure to ionizing radiation. In another embodiment, the compositions described herein are administered following the exposure to ionizing radiation.

By “administration prior to” is meant administration of a composition of the invention in a therapeutically effective amount before exposure to ionizing radiation (e.g. start of radiation therapy) or before radiation exposure becomes likely (e.g., 4 months prior, 3 months prior, 2 months prior, 1 month prior, 4 weeks prior, 3 weeks prior, 2 weeks prior, 1 week prior, 6 days prior, 5 days prior, 4 days prior, 3 days prior, 2 days prior, 1 day prior, less than 24 hours prior (e.g., less than 23, 20, 19, 18, 17, 16, 15, 10, 9, 8, 7, 6, 5, 4, 3, 2 hours, or 1 hour) to the start of radiation therapy or possible radiation exposure).

By “administration concurrently with” is meant administration of a composition of the invention in a therapeutically effective amount with the start of radiation exposure, for example radiation therapy, (e.g., less than 2, 6, 12, 18, or 24 hours after the radiation exposure). Alternatively, “administration concurrently with” can mean between the first and subsequent radiation exposures (e.g. between the first and second doses of chemotherapy or radiation therapy). In one embodiment, administration of a composition of the invention in a therapeutically effective amount starts prior to radiation exposure and is continued concurrently with radiation exposure, or while the possibility of radiation exposure persists.

Examples of subjects who may be in need of radioprotection or radiation mitigation according to the methods of this invention include but are not limited to, patients who are exposed to radiation (e.g., proton radiation, photon radiation) as part of therapeutic regimen (e.g., cancer patients who require radiation therapy), subjects who are exposed to radiation for to diagnose a disease or condition (e.g., subjects receiving dental or bone X-rays, patients receiving PET scans, CT scans and the like). Examples of subjects who may be in need of radioprotection or radiation mitigation according to the methods of this invention also include those who may be exposed to radiation as a result of their profession or life style choices (e.g., airplane flight crews or other frequent air travelers, and even space travelers, who are exposed to higher than average radiation levels; laboratory technicians and other workers; or those exposed through the use of electronic devices) or those exposed to accumulations of radon (e.g., accumulations in dwellings or mines) or outdoor workers or sunbathers exposed to natural radiation from the sun. Other subjects who may be in need of radioprotection according to the methods of this invention include those who are accidentally exposed to radiation, such as leaks or spills, (e.g., nuclear reactor leaks or accidents or laboratory spills). Also contemplated are those exposed to radiation from the detonation of a nuclear warhead, as a result of war or terrorism. Additional subjects encompassed are those who are exposed to a terrorist's detonation of conventional explosives that disperse radioactive materials.

The term “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In addition to humans, the subject may include dogs, cats, pigs, cows, sheep, goats, horses, buffalo, ostriches, guinea pigs, rats, mice, birds (e.g., parakeets) and other wild, domesticated or commercially useful animals (e.g., chicken, geese, turkeys, fish). The term “subject” does not exclude an individual that is normal in all respects. The term “subject” includes, but is not limited to, a human in need of therapy for, or susceptible to, a condition or its sequelae.

Any patent, patent application publication, or scientific publication, cited herein, is incorporated by reference herein in its entirety.

In the following examples, numerous specific details are set forth in order to provide a thorough understanding of the invention. However, it will be understood by those skilled in the art that this invention may be practiced without these specific details. In other instances, well-known methods, procedures, and components have not been described in detail so as not to obscure this invention. Thus, these examples should in no way be construed, as limiting the broad scope of the invention.

### EXAMPLES

### LGM2605 Mitigates Cardiopulmonary Deterioration from Radiation Exposure in Mice

Mice (male, C57/LJ) were exposed to thoracic radiation and evaluated for multiple cardio-pulmonary measurements such as arterial oxygen saturation and pulse distention using non-invasive Pulse Oximetry (MouseOx non-invasive vital signs monitor STARR Life Sciences Corp., Oakmont, Pa.).

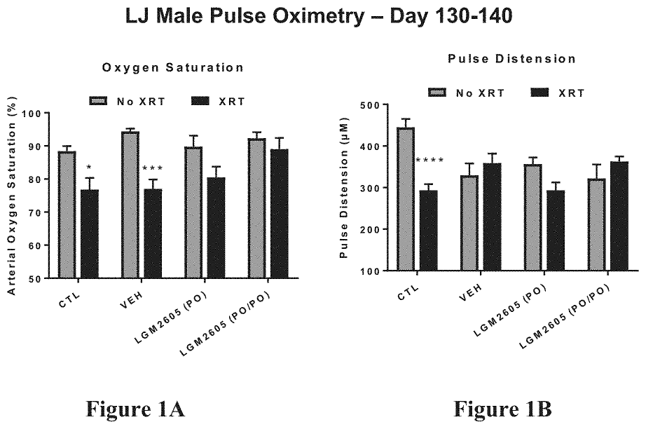

Pulse Distention is the amplitude of the cardiac light absorption signals. Pulse Distention is a hemodynamic measurement obtained by the pulse oximetry software, a measure of the local blood flow (carotid arteries) at the sensor location. Results show that LGM2605 given just early post radiation exposure (daily for 3 weeks) or more prolonged (daily for 3 months), improved arterial blood oxygenation as compared to irradiated controls and vehicle (FIG. 1A). Importantly, pulse distention which decreased significantly in irradiated controls, remained at baseline levels with LGM2605 at either administration regimens (FIG. 1B). This could indicate that LGM2605 helps reduce downstream effects of inflammation in tissues other than the lung.

Significance of findings: These values could translate to physiologic estimates of flow in a cardio-pulmonary circuit. Radiation exposure of lung/heart tissues (just as it does in pulmonary parenchyma) alters the dynamic equilibrium of tissue types within blood vessel walls. Arteries have interwoven cell types ranging from smooth muscle to fibroblast to epithelial. Radiation stimulates inflammatory pathways leading to scars in areas of wall injury. The wall then becomes less compliant, less distensible due to a change in prevailing tissue type from highly dynamic muscle cells to rigid less distensible collagen scars. Physiologically, this would be reflected in a decrease in pulse distension as seen in irradiated control mice, or irradiated mice given vehicle. Each bolus of blood through that given area of vessel scarred by radiation would distend the blood vessel less than uninjured blood vessel because scarred wall is less compliant than is the native uninjured vessel. Pulse distention could serve as a non-invasive surrogate for flow and derivation of resistance within the cardiopulmonary and perhaps systemic circulations.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method for treating or preventing radiation-associated vascular injury in a subject in need thereof, the method comprising: administering to said subject an effective amount of secoisolaricirecinol diglucoside (SDG), an analog thereof, a stereoisomer thereof, or a combination thereof, thereby treating said radiation-induced vascular damage in said subject.

2. The method of claim 1, wherein said SDG is (S,S)-SDG.

3. The method of claim 1, wherein said SDG is (R,R)-SDG.

4. The method of claim 1, wherein said SDG is a synthetic SDG.

5. The method of claim 1, wherein said SDG is an SDG analog.

6. The method of claim 1, wherein said step of administering is performed orally.

7. The method of claim 1, wherein said SDG is administered at a concentration from about 1 nanomolar (nM) to about 1 molar (M).

8. The method of claim 1, wherein the subject is a human subject.

9. The method of claim 1, wherein said radiation is proton radiation.

10. The method of claim 1, wherein said radiation is associated with a medical treatment.

11. The method of claims 1, wherein the subject has been or will be exposed to radiation as part of a therapeutic procedure.

12. The method of claim 1, wherein the subject is a cancer patient and said radiation is associated with a radiation cancer therapy.

13. The method of claim 12, wherein the cancer patient is a lung cancer patient.

14. The method of claim 1, wherein the subject has been or will be exposed to radiation as part of a diagnostic procedure.

15. The method of claim 14, wherein the diagnostic procedure is a dental or bone X-ray.

16. The method of claim 14, wherein the diagnostic procedure is a PET or CT scan.

17. The method of claim 1, wherein the subject has been accidentally exposed to radiation.

18. The method of claim 1, wherein the subject has been or will be exposed to radiation as part of their occupation.

19. The method of claim 18, wherein the subject's occupation is as a laboratory technician.

20. The method of claim 1, wherein the subject has been exposed to radon.

21. The method of claim 1, wherein the subject has been exposed to radiation as a result of terrorism.

22. A method for improving arterial blood oxygenation in a subject who has been or will be exposed to ionizing radiation, the method comprising: administering to said subject an effective amount of secoisolaricirecinol diglucoside (SDG), an analog thereof, a stereoisomer thereof, or a combination thereof, thereby improving arterial blood oxygenation in said subject.

23. The method of claim 22, wherein said SDG is (S,S)-SDG.

24. The method of claim 22, wherein said SDG is (R,R)-SDG.

25. The method of claim 22, wherein said SDG is a synthetic SDG.

26. The method of claim 22, wherein said SDG is an SDG analog.

27. The method of claim 22, wherein said step of administering is performed orally.

28. The method of claim 22, wherein said SDG is administered at a concentration from about 1 nanomolar (nM) to about 1 molar (M).

29. The method of claim 22, wherein the subject is a human subject.

30. The method of claim 22, wherein said ionizing radiation is proton radiation.

31. The method of claim 22, wherein said radiation is associated with a medical treatment.

32. The method of claims 22, wherein the subject has been or will be exposed to radiation as part of a therapeutic procedure.

33. The method of claim 22, wherein the subject is a cancer patient and said radiation is associated with a radiation cancer therapy.

34. The method of claim 33, wherein the cancer patient is a lung cancer patient.

35. The method of claim 22, wherein the subject has been or will be exposed to radiation as part of a diagnostic procedure.

36. The method of claim 35, wherein the diagnostic procedure is a dental or bone X-ray.

37. The method of claim 35, wherein the diagnostic procedure is a PET or CT scan.

38. The method of claim 22, wherein the subject has been accidentally exposed to radiation.

39. The method of claim 22, wherein the subject has been or will be exposed to radiation as part of their occupation.

40. The method of claim 39, wherein the subject's occupation is as a laboratory technician.

41. The method of claim 22, wherein the subject has been exposed to radon.

42. The method of claim 22, wherein the subject has been exposed to radiation as a result of terrorism.

43. A method for protecting vascular tissue against ionizing radiation exposure in a subject in need thereof, the method comprising: administering to said subject an effective amount of secoisolaricirecinol diglucoside (SDG), an analog thereof, a stereoisomer thereof, or a combination thereof, thereby protecting normal vascular tissue against ionizing radiation exposure in said subject.

44. The method of claim 43, wherein said SDG is (S,S)-SDG.

45. The method of claim 43, wherein said SDG is (R,R)-SDG.

46. The method of claim 43, wherein said SDG is a synthetic SDG.

47. The method of claim 43, wherein said SDG is an SDG analog.

48. The method of claim 43, wherein said step of administering is performed orally.

49. The method of claim 43, wherein said SDG is administered at a concentration of about 1 nanomolar (nM) to about 1 molar (M).

50. The method of claim 43, wherein the subject is a human subject.

51. The method of claim 43, wherein said ionizing radiation is proton radiation.

52. The method of claim 43, wherein said radiation is associated with a medical treatment.

53. The method of claims 43, wherein the subject has been or will be exposed to radiation as part of a therapeutic procedure.

54. The method of claim 43, wherein the subject is a cancer patient and said radiation is associated with a radiation cancer therapy.

55. The method of claim 54, wherein the cancer patient is a lung cancer patient.

56. The method of claim 43, wherein the subject has been or will be exposed to radiation as part of a diagnostic procedure.

57. The method of claim 56, wherein the diagnostic procedure is a dental or bone X-ray.

58. The method of claim 56, wherein the diagnostic procedure is a PET or CT scan.

59. The method of claim 43, wherein the subject has been accidentally exposed to radiation.

60. The method of claim 43, wherein the subject has been or will be exposed to radiation as part of their occupation.

61. The method of claim 60, wherein the subject's occupation is as a laboratory technician.

62. The method of claim 60, wherein the subject has been exposed to radon.

63. The method of claim 43, wherein the subject has been exposed to radiation as a result of terrorism.

64. A method for reducing or preventing ionizing radiation-associated blood vessel wall scarring in a subject in need thereof, the method comprising: administering to said subject an effective amount of secoisolaricirecinol diglucoside (SDG), an analog thereof, a stereoisomer thereof, or a combination thereof, thereby reducing or preventing ionizing radiation-associated blood vessel wall scarring in said subject.

65. The method of claim 64, wherein said scarring is collagen scarring.
